(19) United States
(12) Patent Application Publication
(10) Pub. No.: US 2026/0144668 A1
(43) Pub. Date: May 28, 2026

(54) URINE DISCHARGER DETACHABLY ATTACHED TO UNDERPANTS-TYPE DIAPER

(71) Applicants: Hyeong-Rak CHOI, Incheon (KR); Ho-Seok CHOI, Incheon (KR); Yu-Jin CHOI, Incheon (KR); Ho-Chul CHOI, Incheon (KR)

(72) Inventors: Hyeong-Rak CHOI, Incheon (KR); Ho-Seok CHOI, Incheon (KR); Yu-Jin CHOI, Incheon (KR); Ho-Chul CHOI, Incheon (KR)

(21) Appl. No.: 19/105,091

(22) PCT Filed: Aug. 24, 2023
(86) PCT No.: PCT/KR2023/012527

§ 371 Date: Feb. 20, 2025

(30) Foreign Application Priority Data
Aug. 30, 2022 (KR) 1020220108956

(51) Int. Cl. A61F 5/451; A61F 5/44; A61F 13/45; A61F 13/49; A61F 13/496

(52) U.S. Cl. CPC A61F 5/451; A61F 5/4401; A61F 13/49011; A61F 13/49017; A61F 13/496; A61F 2013/4506

(57) ABSTRACT

Disclosed is a urine discharger that is attached to an underpants-type diaper for catching feces and is detachably coupled to the underpants-type diaper configured to discharge urine separately rather than absorbing urine into the diaper. The urine discharger detachably coupled to the underpants-type diaper, according to the present invention, comprises: a wearing unit of which one end is open so as to be in close contact to the periphery of a patient's genitals, has an outlet formed at the other end thereof, and has a downward funnel-shaped hole formed therein; a hose that has one end connected to the outlet; and a urine bag that has an inflow pipe connected to the other end of the hose and an outflow pipe located opposite to the portion at which the inflow pipe is located, and temporarily stores urine excreted by the patient.

### TECHNICAL FIELD

The present invention relates to a urine discharger that collects urine from a disabled person or a seriously ill patient (hereinafter referred to as “patient”) who cannot get out of bed, and more particularly, to a urine discharger detachably coupled to underpants-type diaper that is attached to an underpants-type diaper for catching feces and is detachably coupled to the underpants-type diaper configured to discharge urine separately rather than absorbing urine into the diaper.

### BACKGROUND

In the case of patients who are unable to get out of bed, they cannot get up and dispose of urine and feces themselves, and thus wear diapers or wear urine dischargers.

However, wearing a diaper for a long period of time can lead to problems such as bedsores caused by accumulated urine, and if diapers are changed frequently, the burden on the caregiver and the cost involved increase.

As a result, various types of urine dischargers have been proposed, but conventional urine dischargers cannot process the patient's feces together, and due to structural problems of the urine discharger itself, it is not possible to wear a diaper and an urine discharger together.

### PRIOR ART LITERATURE

Korean Unexamined Utility Model Publication No. 20-1998-063195 (Nov. 16, 1998)

Korean Unexamined Patent Publication No. 10-2018-0112260 (Oct. 12, 2018)

### DETAILED DESCRIPTION OF THE INVENTION

### Technical Problem

It is an object of the present invention to provide a urine discharger detachably coupled to underpants-type diaper that is detachably coupled to the underpants-type diaper for catching feces and is detachably coupled to the underpants-type diaper configured to discharge urine separately rather than absorbing only urine into the diaper, thereby minimizing waste of resources and care service of caregivers.

### Technical Solution

In order to achieve the above object, according to the present invention, there is provided a urine discharger detachably coupled to underpants-type diaper, comprising: a wearing unit of which one end is open so as to be in close contact to the periphery of a patient's genitals, has an outlet formed at the other end thereof, and has a downward funnel-shaped hole formed therein; a hose that has one end connected to the outlet; and a urine bag that has an inflow pipe connected to the other end of the hose and an outflow pipe located opposite to the portion at which the inflow pipe is located, and temporarily stores urine excreted by the patient, wherein the wearing unit has: a fixed wearing unit that has both ends open, one end having a rounded close contact portion formed so as to be in close contact with the periphery of the patient's genitals, and has a wing part protrusively forming from the outer circumference between both the ends so as to be detachably coupled to the underpants-type diaper; and a rotatable wearing unit that has both ends open, one end being rotatably coupled to the other end of the fixed wearing unit so as to rotate left and right, has an outlet formed at the other end thereof, and has a weight receiving part installed at a lower portion thereof.

In the urine discharger detachably coupled to underpants-type diaper, the wing part is formed with a round cross section to fit the periphery of the patient's genitals.

In the urine discharger detachably coupled to underpants-type diaper, the wing part is installed with a Velcro on the inner side surface for detachably connecting to the underpants-type diaper.

The urine discharger detachably coupled to underpants-type diaper further comprises at least one close contact type elastic band, one end of which is first connected to the lower end of the wing part, and the other end of which is equipped with a Velcro for attachment to a rear torso area of the underpants-type diaper.

In the urine discharger detachably coupled to underpants-type diaper, the underpants-type diaper has a coupling hole formed corresponding to at the outer periphery of the fixed wearing unit where the wing part is formed at the periphery of a patient's genitals, the outside of the coupling hole is formed with a coupling part in which velour for Velcro connection are fixedly coupled by backstitching or adhesive, and the left and right sides of a rear torso area is formed with a fastening part in which the other end of the close contact type elastic band and the velour for Velcro coupling are fixedly coupled by backstitching or adhesive.

### BRIEF DESCRIPTION OF THE DRAWINGS

FIG. 1 is a perspective view showing the overall configuration of a urine discharger 1 detachably coupled to underpants-type diaper according to the present invention.

FIG. 2a is a front view of a male wearing unit 10a, FIG. 2b is a perspective view of a male wearing unit 10a viewed from above, and FIG. 2c is a perspective view of a male wearing unit 10a viewed from below.

FIG. 3a is a front view of a female wearing unit 10b, FIG. 3b is a perspective view of a female wearing unit 10b viewed from above, and FIG. 3c is a perspective view of a female wearing unit 10b viewed from below.

FIG. 4a is a conceptual diagram showing the configuration of the inner surface of an underpants-type diaper 3 detachably coupled to a urine discharger 1 according to the present invention, and FIG. 4b is a conceptual diagram showing the configuration of the outer surface detachably coupled to a urine discharger 1 according to the present invention.

FIG. 5 is an illustrative diagram showing the state in which a wearing unit 10 of a urine discharger 1 according to the present invention is combined with a underpants-type diaper 3.

### DETAILED DESCRIPTION OF THE EMBODIMENTS

Hereinafter, preferred embodiments of a urine discharger detachably coupled to underpants-type diaper according to the present invention will be described in detail with reference to the accompanying drawings.

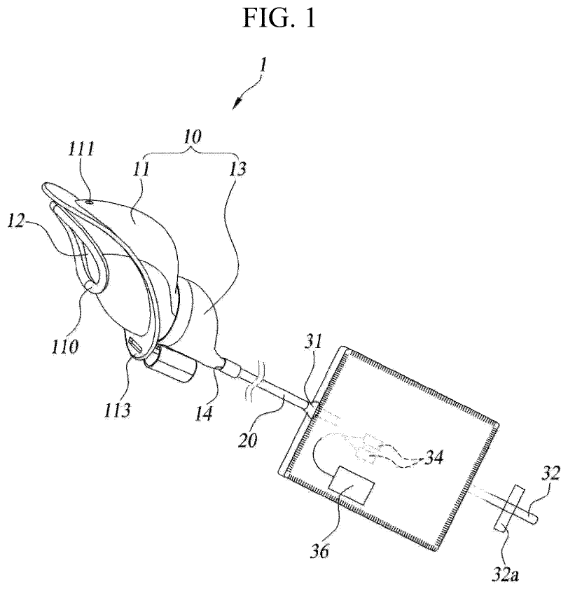

Referring to FIG. 1, the urine discharger 1 according to the present invention comprises a wearing unit 10, a hose 20, and a urine bag 30.

The wearing unit 10 is detachably coupled to the underpants-type diaper 3 and is worn by the patient, wherein a funnel-shaped hole 12 is formed in the part facing the genitals of the male and female patient. The hole 12 formed in the wearing unit 10 is configured such that the outlet 14 faces downward when the patient wearing it lies down.

The wearing unit 10 comprises a fixed wearing unit 11 that is detachably coupled to the underpants-type diaper 3 and fixed to the patient's body, and a rotatable wearing unit 13 that is coupled to the fixed wearing unit 11 so as to rotate left and right.

The fixed wearing unit 11 is open at both ends, and the internal area becomes part of the hole 12. One end of the fixed wearing unit 11 is open to form a close contact part 110 that is in close contact with the patient's genital area, and the other end is rotatably coupled to the rotatable wearing unit 13. Since the close contact part 110 is a part that directly touches the patient's skin, it be preferably rounded with silicone material.

The upper part of the fixed wearing unit 11 is formed with one or more air holes 111, so that even if the patient wears the wearing unit 11 for a long time, the patient's skin prevents occurrence of skin diseases such as bedsores.

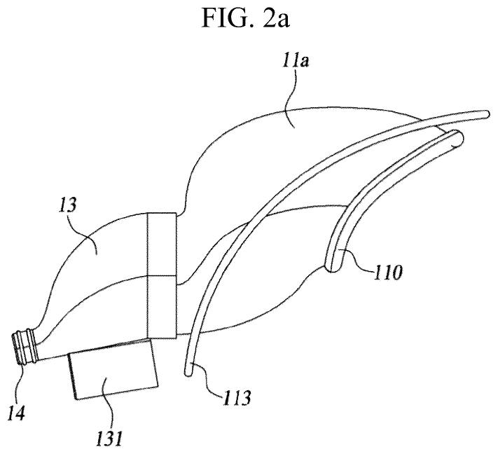

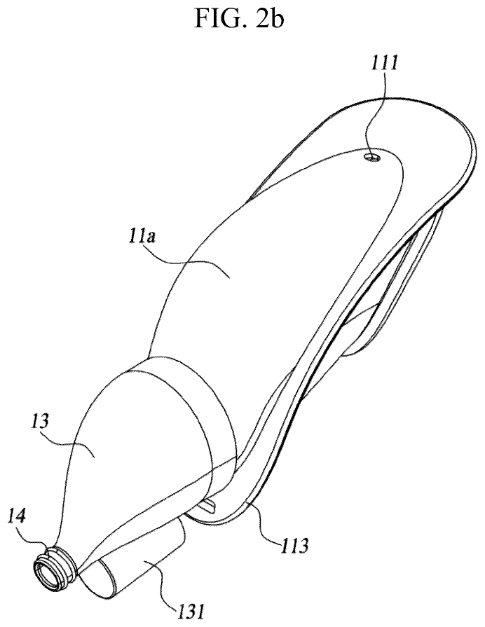

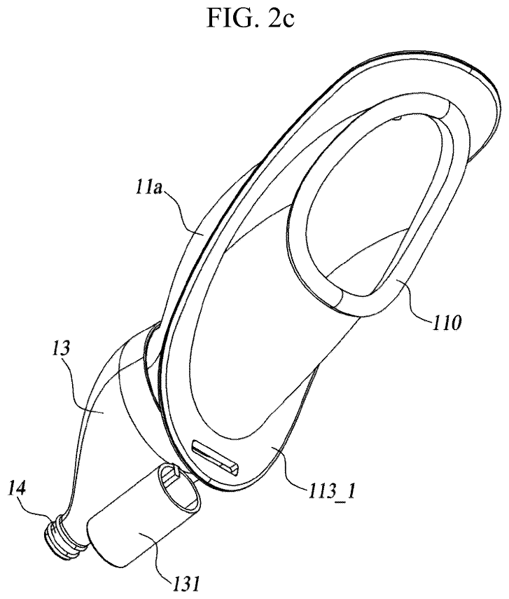

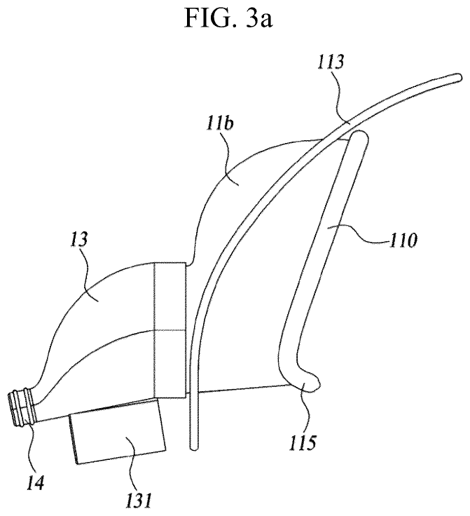

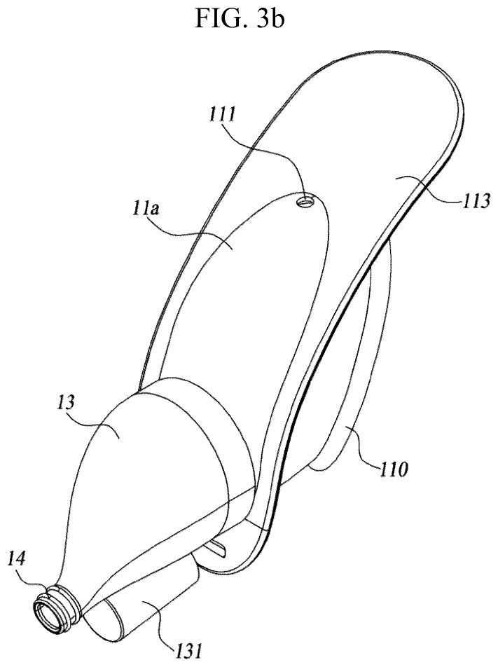

As shown in FIG. 2a to FIG. 3c, it is preferable that the shape of the fixed wearing unit 11 be slightly different depending on the gender of the patient wearing it.

In the case of the male wearing unit 10a shown in FIGS. 2a to 2c, the fixed wearing unit 11a is manufactured so that the funnel-shaped hole 12 has sufficient space even in a state in which the male patient's genitals are accommodated therein. Therefore, the male patient's genitals can be comfortably erected or relaxed inside the funnel-shaped hole 12.

In the case of the female wearing unit 10b shown in FIGS. 3a to 3c, the fixed wearing unit 11b may have a hole 12 that is relatively smaller in size than that of the male wearing unit, and the hole formed in the close contact part 110 that is close to the periphery of the genitals is relatively larger in size, and a protruding part 115 is preferably formed to support the perineum of the patient.

The outer periphery between the two ends of the fixed wearing unit 11 is protrusively formed with a wing part 113 and is detachably coupled to the underpants-type diaper 3.

The wing part 113 is preferably formed with a round cross section so that it can comfortably support the patient's genital area. In addition, the wing part 113 is installed with a Velcro (crochet corresponding to a hook among Velcro) on the inner side surface 113-1 for detachably coupling to the underpants-type diaper.

The rotatable wearing unit 13 has both ends open, one end being rotatably coupled to the other end of the fixed wearing unit 11 so as to rotate left and right to a desired extent. Further, a weight receiving part 131 is installed at a lower part of the rotatable wearing unit 13. Therefore, when a patient lies on his/her side, even if the fixed wearing unit 11 is fixed to the patient's body, the rotatable wearing unit 13 is automatically controlled so that the outlet 14 of the wearing unit 10 faces stably downward by the weight.

The wearing unit 10 is preferably made of soft or hard PVC.

The wearing unit 10 may be further installed with one or more close contact type elastic bands 15, one end of which is coupled to the lower end of the wing part 113. The other end of the close contact type elastic band 15 is equipped with a Velcro (crochet equivalent to a hook in Velcro) for attaching to a rear torso area of the underpants-type diaper 3.

When two close contact type elastic bands 15 are installed, they can be attached to a rear torso area of the underpants-type diaper 3 in a ‘V’ shape as a whole. The close contact type elastic bands 15 pressurize the wearing unit 10 of the urine discharger 1 to be more closely contacted to the patient's body (especially the lower part of the genitals) by elasticity, thereby preventing the patient's urine from leaking out of the wearing unit 10.

The hose 20 is a passage for sending the patient's urine from the wearing unit 10 to the urine bag 30.

One end of the hose 20 is connected to the outlet 14 of the hole 12. When the patient discharges urine, the diameter of the hose 20 is determined to be a sufficiently large value of 10 to 20 mm so that the urine can be discharged directly without filling the hole 12. Therefore, no matter how strongly the patient discharges urine, the discharged urine can be discharged immediately through the hose 20 without overflowing from the funnel-shaped hole 12, or without being heavily adhered to the patient's skin, or without being absorbed into the underpants-type diaper 3. In the present invention, the length of the hose 20 is not limited. Therefore, the length of the hose 20 can be adjusted appropriately according to the convenience of use.

In the urine discharger 1 according to the present invention, one end of the hose 20 may be detachably connected to the outlet 14 of the hole by elastic coupling or screw coupling, etc.

The urine bag 30 is a bag that temporarily stores urine discharged by the patient, 6 and is preferably made of PE (Polyethylene) or the like. An inflow pipe 31 is installed at one end of the urine bag 30, and an outflow pipe 32 is installed at the other end of the portion at which the inflow pipe 31 is located.

The inflow pipe 31 of the urine bag 30 is coupled to the other end of the hose 20, and may be detachably connected by elastic coupling or screw coupling, etc.

It is preferable that the outflow pipe 32 of the urine bag 30 is equipped with a switch 32a.

A water sensor 34 is installed inside the urine bag 30 to detect the presence or absence of urine, and an alarm 36 is installed outside the urine bag 30 and passes through the urine bag 30 to be electrically connected to the water sensor 34, and an alarm sound is generated when the water sensor 34 detects urine. The alarm 36 also has its own small battery to supply power required for the operation of the water sensor 34 and the alarm 36.

According to another embodiment of the present invention, the result value of the water sensor 34 is transmitted to the mobile phone of the guardian or caregiver via Bluetooth or the like, and the result value can be output as an alarm sound or the like via an app on the mobile phone. Therefore, the guardian or caregiver can easily check whether urine is discharged by the alarm sound output via the alarm 36 or mobile phone without looking inside the urine bag 30.

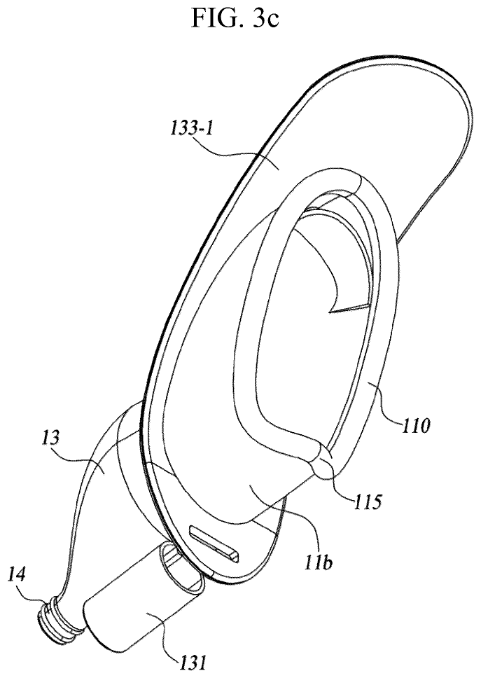

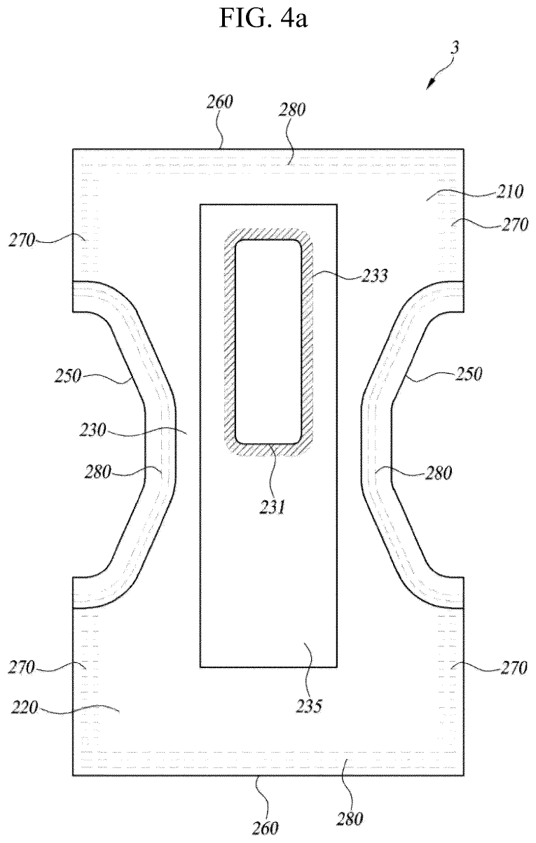

FIG. 4a is a conceptual diagram showing the configuration of the inner surface of an underpants-type diaper 3 detachably coupled to a urine discharger 1 according to the present invention, and FIG. 4b is a conceptual diagram showing the configuration of the outer surface detachably coupled to a urine discharger 1 according to the present invention.

First, the underpants-type diaper 3 is basically made of non-woven fabric, and may be coated with an impermeable plastic film on the outside. However, the scope of the present invention is not limited by the material of the underpants-type diaper 3.

Referring to FIGS. 4a and 4b, the underpants-type diaper 3 is composed of a front torso area 210, a rear torso area 220, and a crotch area 230 located between the two areas. The front torso area 210 and the rear torso area 220 are joined to overlap parts of their side edges, so that a pair of leg openings 250 are formed on the left and right side of the side edges that are not joined, and a waist opening 260 is formed on the upper side. At this time, the front torso area 210 and the rear torso area 220 have Velcro tapes 270 joined to parts of their side edges, so that the pant-type diaper 3 in a flat state may be changed to an underpants-type state to fit the patient, or may be change to a flat state by loosening the Velcro tapes 270 even when the underpants-type diaper 3 is removed from the patient. Unlike the same, it goes without saying that, instead of joining the Velcro tapes 270 to the side edges of the front torso area 210 and the rear torso area 220, the corresponding parts may be fixed and fastened to each other to manufacture and provide the underpants-type diaper from the beginning.

At least one elastic band 280 is joined in a stretched state to the waist opening 260 and leg openings 250 of the underpants-type diaper 3. The elastic band joined to the waist opening 260 serves to fix the underpants-type diaper 3 to the patient's body, and the elastic band attached to the leg openings 250 prevents urine and feces discharged from the patient's body from leaking to the outside of the underpants-type diaper 3.

A pad 235 made of cotton or pulp material is joined to the crotch area 230 of the underpants-type diaper 3, and the pad 235 comes into direct contact with and absorbs moisture generated by the patient's feces and some urine that was not completely discharged through the urine discharger 1.

The crotch area 230 of the underpants-type diaper 3 is formed with a coupling hole 231 corresponding to the outer circumference of a fixed wearing unit 11 formed with a wing part 113 of a urine discharger 1 around the patient's genital area. Around the coupling hole 231, a coupling part 233 is formed in which a soft material fabric is fixedly coupled on the inside and velour for Velcro fastening are fixedly coupled on the outside by backstitching or adhesive.

The edge forming the inner diameter of the coupling hole 231 is rounded with silicone, so that it can maintain airtightness when coupled with the wing part 113 of the urine discharger 1 through the coupling hole 231.

The coupling part 233 of the underpants-type diaper 3 is coupled to the wing part 113 of the urine discharger 1 to support the wearing unit 10, and at this time, the contact part 110 of the fixed wearing unit 11 is positioned slightly apart from the wing part 113. Therefore, when the patient wears the underpants-type diaper 3 coupled to the wearing unit 10, the close contact part 110 rounded with a silicone material strongly presses the patient's genitals, making it easy to contact the skin while maintaining airtightness.

Further, referring to FIG. 4b, the left and right sides of the rear torso area 220 of the underpants-type diaper 3 are formed with a fastening part 221 in which the other end of the close contact type elastic band 15 and the velour for Velcro coupling are fixedly coupled by backstitching or adhesive.

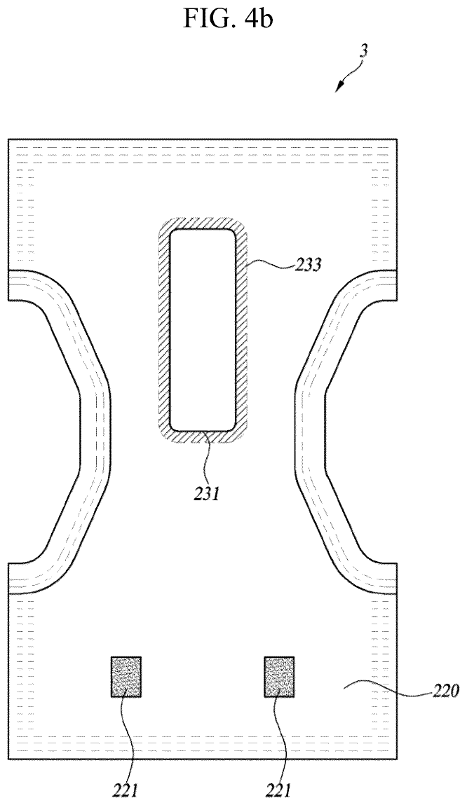

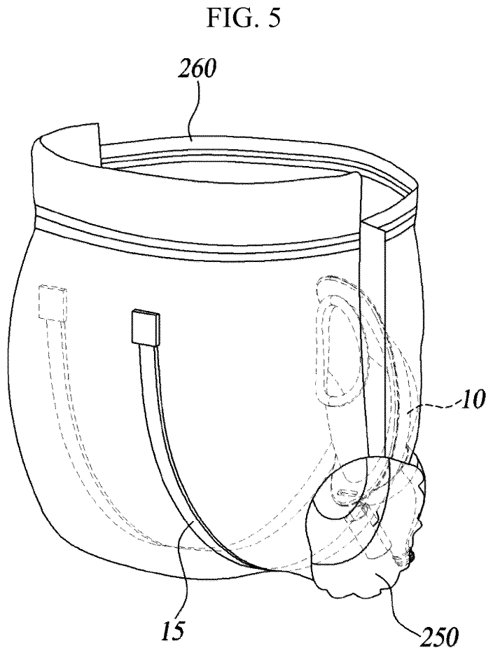

Next, the process of collecting urine and faces from a patient using a urine discharger 1 detachably coupled to an underpants-type diaper according to the present invention will be described with reference to FIGS. 1 to 5.

First, the upper part of the fixed wearing unit 11 of the urine discharger 1 is fitted from the outside to the inside of the coupling hole 231 formed in the underpants-type diaper 3, and the inner side surface 113-1 of the wing part 113 is velcro-coupled to the coupling part 233 around the outside of the coupling hole 231 of the underpants-type diaper 3. Then, the underpants-type diaper 3 equipped with the wearing unit 10 is put on the patient. At this time, in the underpants-type diaper 3 in a flat state, the velcro tape 270 joined to the side edges of the front torso area 210 and the rear torso area 220 are joined to each other and thus can be easily converted to the underpants type to fit the patient. After that, when the funnel-shaped hole 12 of the wearing unit 10 is positioned to face the patient's genitals, the hole 12 formed in the wearing unit 10 is fixed with the outlet 14 facing downward in a state where the patient wearing it lies down.

Next, if the Velcro attached to the close contact type elastic band 15 installed at a lower end of the wing part 113 of the wearing unit 10 is coupled to the fastening part 221 formed in the rear torso area 220 of the underpants-type diaper 3, the wearing unit 10 of the urine discharger 1 is further closed contacted to the patient's body by the ‘V’-shaped close contact type elastic band 15.

The patient can wear the underpants-type diaper 3 equipped with a wearing unit 10 even in normal times, and the funnel-shaped hole 12 has sufficient extra space even in a state where the genitals of the male patient are accommodated therein, and one or more air holes 111 are formed in the upper part of the wearing unit 10 to allow air circulation. Therefore, even if the patient wears the underpants-type diaper 3 equipped with a wearing unit 10, skin diseases such as bedsores do not occur on the patient's skin, and the genitals of the male patient can comfortably be erected or maintained in a relaxed state inside the funnel-shaped hole 12. In the case of the female wearing unit 10b, the size of the hole 12 can be formed relatively smaller in side than that of the male wearing unit 10a.

If the patient is not lying upright but lying on the side, the fixed wearing unit 11 is fixed to the patient's body and even if it turns to the side, the rotatable wearing unit 13 is automatically rotated by the weight received in the weight receiving part 131, so that the outlet 14 of the wearing belt 10 is stably controlled downward.

When the patient feels the need to urinate and the guardian or caregiver wants to collect the patient's urine, it is sufficient to simply connect the hose 20 with the urine bag 30 to the w earing unit 10. The patient can then urinate comfortably while lying down.

Further, depending on the situation, the hose 20 with the urine bag 30 attached may be left in a state of being continuously connected to the wearing unit 10.

When urine is discharged into the urine bag 30, a water sensor 234 installed inside the urine bag 30 detects that urine has been discharged, and transmits the result to the alarm 36 installed outside the urine bag 30, and the alarm 36 generates an alarm sound according to the detection result transmitted from the water sensor 34.

When the patient finishes urinating, the guardian or caregiver detaches the hose 20 equipped with the urine bag 30 from the patient's wearing unit 10, and then operates the switch 32a on the outflow pipe 32 of the urine bag 30 as shown in FIG. 3 to discharge the urine inside, and reattaches the hose 20 to the belt 10.

According to such a process, the patient does not have to expose his/her genitals to the guardian or caregiver every time he/she urinates, and the wearing unit 10 worn by the patient is not in close contact with the patient's skin except for certain areas, preventing the patient from having to endure the side effects of skin disease.

When a patient defecates while wearing the underpants-type diaper equipped with a urine discharger according to the present invention, the pad 235 joined to the crotch area 230 of the underpants-type diaper 3 absorbs moisture contained in the patient's feces. After that, the guardian or caregiver separates the wearing unit 10 from the underpants-type diaper 3, loosens the Velcro tape 270 to switch the underpants-type diaper 3 to a flat state, and then disposes of the patient's urine and feces together with the underpants-type diaper 3, and then can put a new underpants-type diaper 3 on the patient.

Since feces discharge count is usually significantly lower than that of urine, the underpants-type diaper 3 is wasted by urine, and the trouble of the guardian or caregiver having to change the underpants-type diaper 3 is greatly reduced. Further, from the patient's perspective, the patient's genitals are only exposed when defecation is performed, so the patient's sense of shame and discomfort can be significantly reduced, and the patient does not need to wear a diaper that has absorbed a large amount of urine for a long period of time, so that the possibility of developing skin diseases such as bedsores is significantly reduced.

### industrial Applicability

According to the present invention, since feces discharge count is usually significantly lower than that of urine, the underpants-type diaper is wasted by urine, and the trouble of the guardian or caregiver having to change the underpants-type diaper is greatly reduced. Further, from the patient's perspective, the patient's genitals are only exposed when defecation is performed, so the patient's sense of shame and discomfort can be significantly reduced, and the patient does not need to wear a diaper that has absorbed a large amount of urine for a long period of time, so that the possibility of developing skin diseases such as bedsores is significantly reduced.

In addition, the wearing unit can be automatically controlled to keep the outlet facing downwards even if the patient changes position, and guardians or caregivers can easily check whether urine has been discharged by an alarm sound, without having to check inside the urine bag where urine is collected.

1. A urine discharger detachably coupled to underpants-type diaper, comprising:
a wearing unit of which one end is open so as to be in close contact to the periphery of a patient's genitals, has an outlet formed at the other end thereof, and has a downward funnel-shaped hole formed therein;
a hose that has one end connected to the outlet; and
a urine bag that has an inflow pipe connected to the other end of the hose and an outflow pipe located opposite to the portion at which the inflow pipe is located, and temporarily stores urine excreted by the patient,
wherein the wearing unit has:
a fixed wearing unit that has both ends open, one end having a rounded close contact portion formed so as to be in close contact with the periphery of the patient's genitals, and has a wing part protrusively forming from the outer circumference between both the ends so as to be detachably coupled to the underpants-type diaper; and
a rotatable wearing unit that has both ends open, one end being rotatably coupled to the other end of the fixed wearing unit so as to rotate left and right, has an outlet formed at the other end thereof, and has a weight receiving part installed at a lower portion thereof.

2. The urine discharger detachably coupled to underpants-type diaper according claim 1,
wherein the wing part is formed with a round cross section to fit the periphery of the patient's genitals.

3. The urine discharger detachably coupled to underpants-type diaper according to claim 2,
wherein the wing part is installed with a Velcro on the inner side surface for detachably connecting to the underpants-type diaper.

4. The urine discharger detachably coupled to underpants-type diaper according to claim 2,
further comprising at least one close contact type elastic band, one end of which is first connected to the lower end of the wing part, and the other end of which is equipped with a Velcro for attachment to a rear torso area of the underpants-type diaper.

5. The urine discharger detachably coupled to underpants-type diaper according to claim 4,
wherein the underpants-type diaper has a coupling hole formed corresponding to at the outer periphery of the fixed wearing unit where the wing part is formed at the periphery of a patient's genitals,
the outside of the coupling hole is formed with a coupling part in which velour for Velcro connection are fixedly coupled by backstitching or adhesive, and
the left and right sides of a rear torso area is formed with a fastening part in which the other end of the close contact type elastic band and the velour for Velcro coupling are fixedly coupled by backstitching or adhesive.
